(19) United States
(12) Patent Application Publication
(10) Pub. No.: US 2005/0085723 A1
(43) Pub. Date: Apr. 21, 2005

(54) Radiolucent medical devices with radiopaque markers

(76) Inventor: Joel Huebner, Jacksonville, FL (US)

Correspondence Address: Mark J. Young, Suite 227, 9951 Atlantic Blvd., Jacksonville, FL, 32225, US

(21) Appl. No.: 10/958,099

(22) Filed: Oct. 4, 2004

Related U.S. Application Data
(60) Provisional application No. 60/509,029, filed on Oct. 4, 2003.

(51) Int. Cl. A61B 19/00

(52) U.S. Cl. 600/431; 128/899

(57) ABSTRACT

A medical device includes a body comprised substantially of radiolucent material and a radiopaque marker. The radiolucent material does not obscure images or radiographs during fluoroscopic visualization. The radiopaque marker is detectable during radiological imaging. The radiopaque marker may produce a readily identifiable image according to the type of medical device. The marker may further be configured to produce a magnetic field or transmit signals for noninvasive detection using a sensor. The field and signals may be unique for each type of medical device.

### RELATED APPLICATION

This application claims priority to U.S. Provisional Application 60/509,029, filed Oct. 4, 2003, the entire contents of which are incorporated by reference herein.

### BACKGROUND OF THE INVENTION

1. Field of the Invention

This invention generally relates to medical devices, and more particularly, to radiolucent medical devices with radiopaque markers.

2. Background Description

Many surgical devices are composed of stainless steel or other radiopaque materials. During surgeries that require fluoroscopic visualization of internal organs or bones, such radiopaque devices (e.g., retractors) must be temporarily removed to avoid obscuring the image. Aside from being inconvenient, removal and subsequent reinsertion consumes valuable time and interferes with the smooth flow of an operation. Fluoroscopic visualization of internal organs before and after removal of radiopaque devices (e.g., retractors) results in a greater number of X-rays, and therefore admitting more radiation to the patient and staff.

Concomitantly, radiolucent devices would not gain widespread acceptance by surgeons due to several shortcomings, the most important of which concerns detectability. A problem faced during surgery, especially in cases involving emergency surgery, obese patients, or unplanned changes in surgical procedures, is the accidental leaving of medical instruments inside surgical patients. Overlooked equipment often necessitates reoperation and can lead to serious problems ranging from organ perforation and blood infection to death. While protocols, such as counting procedures, exist to reduce the risk of overlooking an instrument, mistakes ar bound to happen.

Significantly, overlooked instruments are typically detected by fluoroscopic visualization or radiograph images, in X-rays, or by other radiation imaging techniques. But for the radiopacity of equipment left inside a patient, the overlooked devices may never be detected.

Other shortcomings associated with many radiolucent materials and devices include inferior structural performance (e.g., less rigidity) as compared to conventional radiopaque (e.g., stainless steel) counterparts, and weakening during sterilization.

The invention is directed to overcoming one or more of the problems as set forth above.

### SUMMARY OF THE INVENTION

In one aspect of the invention, a detectable radiolucent medical device is provided. The device includes a medical device body comprised of a radiolucent material. A radiopaque indicator is attached to the body of the device to facilitate detection of the device in radiological imaging, without substantially or materially obscuring images taken in the ordinary course of surgical procedures.

In another aspect of the invention, the radiopaque indicator may produce a detectable field to facilitate detection of the medical device using a detecting means if the medical device is inadvertently left in a patient's body. For example, the radiopaque indicator may be comprised of a magnetic (e.g., ferromagnetic) material. The magnetic material produces a magnetic field which may be detected using a conventional magnetic field sensor, such as.

In a further aspect of the invention, the radiopaque indicator includes a signal transmitter. The transmitter may transmit signals which may be detected using a conventional receiver.

In yet a further aspect of the invention, a system is provided. The system includes a radiolucent medical device with a radiolucent indicator in accordance with the invention. The system further includes a means for noninvasive detection of such a medical device if the device is left within a patient's body.

### BRIEF DESCRIPTION OF THE DRAWINGS

The foregoing and other objects, features and advantages of the present invention will become better understood with reference to the following description, appended claims, and accompanying drawings, where:

FIG. 1 depicts a radiolucent retractor with a radiopaque marker in accordance with an exemplary embodiment of the invention;

FIG. 2 depicts a radiolucent retractor with a radiopaque marker in accordance with an exemplary embodiment of the invention;

FIG. 3 depicts radiolucent forceps with a radiopaque marker in accordance with an exemplary embodiment of the invention;

FIG. 4 depicts a radiolucent retractor with a radiopaque marker in accordance with an exemplary embodiment of the invention;

FIG. 5 depicts a radiolucent retractor with a radiopaque marker in accordance with an exemplary embodiment of the invention; and

FIG. 6 depicts a radiolucent retractor with a radiopaque marker in accordance with an exemplary embodiment of the invention.

### DETAILED DESCRIPTION

A medical device in accordance with an exemplary embodiment of the present invention includes a body comprised substantially of radiolucent material and a radiopaque marker. The medical device may be a retractor, speculum, forceps, holder or some other implement that could benefit from radiolucency.

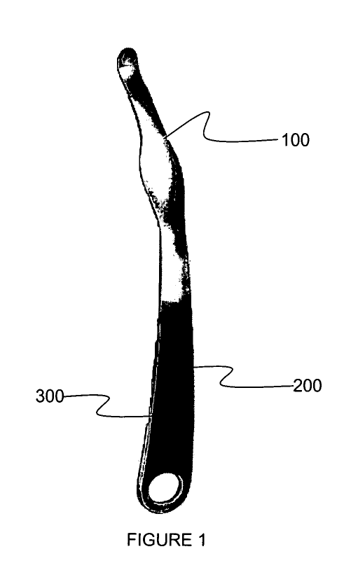

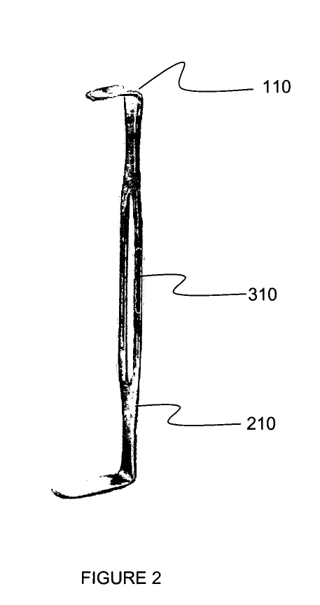

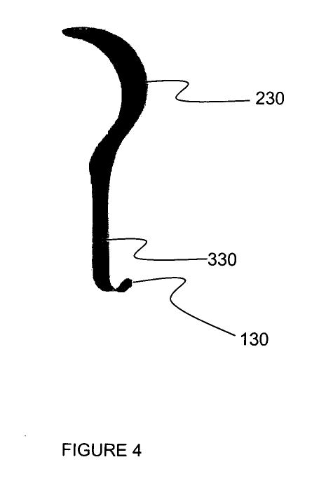

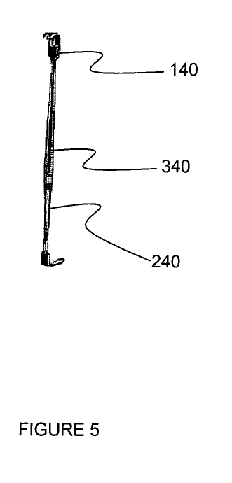

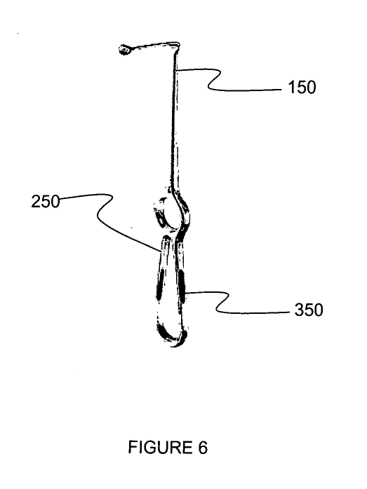

Referring now to FIGS. 1 through 6, various radiolucent medical devices are shown. The body of each device may, for example, include one or more engaging portions 100-150 and one or more handles 200-250 of the device. Each device also includes one or more radiopaque markers 300-350. The markers are located at points which should not substantially obscure X-rays, or other radiological images, produced while the device is used during normal surgical procedures. The body is preferably comprised entirely (or substantially) of a radiolucent material that exhibits adequate structural and thermal properties. The handle may optionally be comprised of the same or a similar radiolucent material or a radiopaque material. Those skilled in the art will appreciate that the type and function of the device will dictate the geometry and desired physical properties of the body and the handle.

Those skilled in the art will also appreciate that the principles of the invention apply to any handheld medical device that might inadvertently be left inside a patient. Thus, the devices illustrated in FIGS. 1 through 6 are provided for exemplary purposes only, and not to limit the scope of the invention in any way.

Preferably the radiopaque material comprising the body is relatively inexpensive and easy to mold or otherwise form into desired shapes, and radiolucent. Additionally, the material must be bio-compatible, i.e., safe for internal use. The material should also be capable of withstanding and retaining mechanical integrity through sterilization processes. Alternatively, the material and manufactured device may be inexpensive enough so that contaminated devices may be disposed.

A variety of biocompatible materials such as Polyphenolsulfone, Polyetherether ketone (PEEK), Ultrapek (polyether ketone ether ketone ketone), Polysulfone, Acetal (Delrin), Polyethylene, Polycarbonate, Polypropylene, Polyetherimide, Polyethersulfone, Polyphenylsufide and derivatives and composites of these materials, optionally involving high strength carbon fibers or glass filaments or other structural additives for stiffening and strengthening. Other materials (e.g., other plastics and polymers) that exhibit bio-compatibility, radiolucency and adequate mechanical properties may be used without departing from the scope of the invention.

In a preferred embodiment, a device in accordance with an exemplary implementation of the present invention includes at least one marker 300-350. The maker may be embedded in the device, attached to the device or otherwise comprise a portion of the device. For convenience, such embedding, attaching or comprising are referred to herein as attaching. A marker so embedded, attached or comprising a part of the device is considered “attached” to the device.

The marker (or indicator) is a radiopaque portion. It may be comprised of steel, stainless steel or some other radiopaque material. Preferably, the marker material is also bio-compatible.

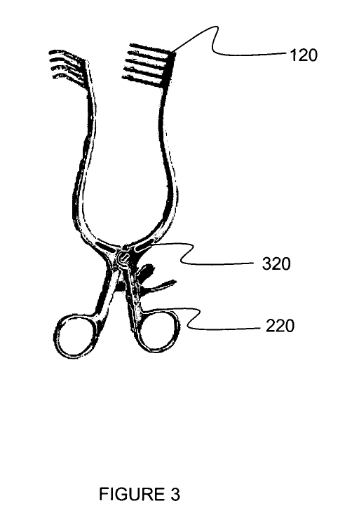

The marker is selectively located so as to reveal the device if it is left in a patient. Fluoroscopic procedures or radiographs will reveal the radiopaque marker 300-350. However, the marker is located so as to not materially obscure operative areas of interest during surgical fluoroscopy and other radiological imaging techniques. By way of example and not limitation, a marker 300-350 may be located near the handle, rather than the engaging end 100-150 (e.g., blade) of a retractor. If a device has a plurality of parts, any one of which may become separated from the others and left within a patient, then a marker 320 and 325 may be included within each part, as shown in FIG. 3.

The geometry and size of the marker 300-350 may vary depending upon the device, manufacturing process and materials. However, the marker should be large enough to readily detect during surgical fluoroscopy and other radiological imaging techniques. Additionally, the marker should have a configuration (e.g., shape) that would not be mistaken for something else. By way of example and not limitation, unnatural (i.e., not occurring naturally) shapes such as stars, triangles, alphanumeric characters, barcodes or a sequence of indicia are preferred, though other shapes and combinations (even natural shapes such as circles) may be used without departing from the scope of the present invention.

Each type of instrument may also have a unique marker. Thus, by way of example, forceps may have a marker that is differently shaped than the marker for a retractor. Thus, if a radiograph reveals a marker, an informed professional can readily determine the associated instrument.

Various techniques may be used to manufacture the device. Of course, the material will limit the types of suitable manufacturing techniques known in the art for the chosen material, such as (for example) injection, compression, blow, or transfer molding. For example, carbon fiber reinforced polyetheretherketone may be injection molded. The marker may be included in the mold or added to the device after molding. Preferably the manufacturing technique is suitable for mass production at relatively low cost per unit, and results in an aesthetically acceptable product with a consistent acceptable quality.

In an alternative implementation, in addition to being radiopaque, the marker may be configured to produce a detectible signal or field. For example, a magnetic (e.g., ferromagnetic or electromagnetic) material may be used to produce a magnetic field that can be detected using conventional magnetic field detection equipment. Permanent magnets are preferred since they are generally inexpensive, highly accessible, easy to use, and have a very long shelf life and usable lifetime. Examples of permanent magnets include flexible ferrite magnets containing magnetic powders impregnated in a binder, alnico magnets, and rare earth magnets. Rare earth magnets are preferred since they provide the strongest and most easily measurable magnetic fields. Examples of rare earth magnets are neodymium-iron-boron magnets and samarium-cobalt magnets.

In one embodiment, a each type of device may be equipped with a magnetic marker that produces a unique detectible magnetic field that is indicative of the type of medical equipment. Illustratively, the marker for a first type of medical device may produce a first type of magnetic field, while the marker for a second type of medical device may produce a second type of magnetic field. Thus, by detecting the type of magnetic field produced, a trained professional can readily determine the associated device.

By way of example and not limitation, a variety of magnetic devices and magnetic field detectors can be used with the present invention. Gaussmeters and magnetometers are examples of suitable detectors. A variety of gaussmeters are available. Such sensors may be used to locally detect weak magnetic fields produced by magnetic markers attached to or embedded in a radiolucent medical device in accordance with the invention.

Using such a sensor, medical staff may quickly scan a patient for any anomalous magnetic fields which would indicate the presence of an overlooked device within the patient. Such scanning is preferably performed before a surgical patient is stitched or stapled closed. Additionally, the scanning is preferably performed in addition to conventional counting processes.

Alternatively, the marker may include a signal transmitter, such as, for example, a battery powered radio frequency (RF) transmitter. Such markers could greatly facilitate detection. Again, at the end of a procedure, but before closing a patient, the patient may be scanned using a tuned RF receiver to determine whether a device or a portion of a device containing a marker has been left in a patient.

In one embodiment, each type of device may be equipped with a transmitter that produces a unique detectible signal that is indicative of the type of medical equipment. Illustratively, the marker for a first type of medical device may produce a first type of signal, while the marker for a second type of medical device may produce a second type of signal. Thus, by detecting the type of signal produced, a trained professional can readily determine the associated device.

Those skilled in the art will appreciate that the invention provides a system for noninvasive detection of radiolucent devices left within a patient. The system includes a radiolucent medical device with a radiolucent indicator. The system further includes a means for noninvasive detection of such a medical device if it is left within a patient's body. The means for noninvasive detection may include fluoroscopic imagers, X-ray imaging devices, or other radiological imaging devices. If the marker is magnetic, the means for noninvasive detection may further include a magnetic field detection device. If the marker includes a signal transmitter, the means for noninvasive detection may further include a signal receiver.

Those skilled in the art will also appreciate that the invention further provides a method for non-invasively detecting a medical device according to the invention that has been left in a patient. The method includes a step of producing a fluoroscopic image, X-ray image, or other radiological image of an area of interest for a patient. The area of interest may be any part of a patient where a medical device may have been left behind, or to which the device may have migrated after a surgical procedure. Such images would reveal the radiopaque marker of a device in the area of interest. If the device includes a magnetic marker, the method may entail scanning a patient for a magnetic field from such a device. If the marker includes a signal transmitter, the method may entail scanning a patient for a signal transmitted from such a device.

While the invention has been described in terms of various embodiments, implementations and examples, those skilled in the art will recognize that the invention can be practiced with modification within the spirit and scope of the appended claims.

1. A handheld medical device comprising:
a medical device body comprised of a radiolucent material; and
a radiopaque indicator attached to said body.

2. A medical device according to claim 1, wherein the radiolucent material includes a material from the group consisting of Polyphenolsulfone, Polyetherether ketone, Polyether ketone ether ketone ketone, Polysulfone, Acetal, Polyethylene, Polycarbonate, Polypropylene, Polyetherimide, Polyethersulfone, and Polyphenylsufide.

3. A medical device according to claim 2, wherein the radiolucent material includes fiber reinforcement.

4. A medical device according to claim 3, wherein the fiber reinforcement includes carbon fiber reinforcement.

5. A medical device according to claim 3, wherein the fiber reinforcement includes glass fiber reinforcement.

6. A medical device according to claim 1, wherein the radiopaque indicator is configured to exhibit an unnatural shape.

7. A medical device according to claim 6, wherein the radiopaque indicator is configured to exhibit a shape indicative of a type of medical device.

8. A medical device according to claim 1, wherein the radiopaque indicator is comprised of a magnetic material.

9. A medical device according to claim 8, wherein the radiopaque indicator comprised of a magnetic material is comprised of a permanent magnetic material from the group consisting of ferrite magnets, alnico magnets, neodymium-iron-boron magnets, and samarium-cobalt magnets.

10. A medical device according to claim 1, wherein the radiopaque indicator is comprised of an electromagnet

11. A medical device according to claim 1, wherein the radiopaque indicator is comprised of an RF signal transmitter.

12. A medical device detection system comprising:
a medical device comprised of
a medical device body comprised of a radiolucent material; and
a radiopaque indicator attached to said body, said radiopaque indicator producing a sensible output; and
a sensing apparatus configured to detect the sensible output produced by radiopaque indicator when said sensing apparatus is placed in proximity to said medical device.

13. A medical device detection system according to claim 12, wherein the sensible output is a magnetic field and the sensing apparatus is a magnetic field sensing apparatus.

14. A medical device detection system according to claim 13, wherein the magnetic field produced is indicative of a type of medical equipment.

15. A medical device detection system according to claim 13, wherein the magnetic field sensing apparatus includes a gauss meter.

16. A medical device detection system according to claim 13, wherein the magnetic field sensing apparatus includes a magnetometer.

17. A medical device detection system according to claim 13, wherein the sensible output is a RF signal and the sensing apparatus is an RF signal sensing apparatus configured to detect an RF signal transmitted by said RF signal transmitter when said RF signal sensing apparatus is placed in proximity to said medical device.

18. A medical device detection system according to claim 18, wherein the RF signal and the sensing apparatus is an RF signal is indicative of a type of medical equipment.

19. A medical device detection methodology comprising:
placing a sensing apparatus adjacent to a patient's body to detect the presence or absence of a medical device within the patient's body, said medical device being adapted to produce sensible output, and said sensing apparatus being adapted to detect said sensible output.

20. A medical device detection methodology according to claim 19, wherein:
the medical device is adapted to produce sensible output from the group consisting of a magnetic field and RF signal.
